(19) United States
(12) Patent Application Publication
(10) Pub. No.: US 2025/0101455 A1
(43) Pub. Date: Mar. 27, 2025

(54) THREE-DIMENSIONAL BIOREACTOR INCLUDING FILLED VOID STRUCTURE

(71) Applicant: Southwest Research Institute, San Antonio, TX (US)

(72) Inventors: Kreg A. ZIMMERN, San Antonio, TX (US); Nicholas P. mcmahon, San Antonio, TX (US); Jian LING, JR., Spring Branch, TX (US)

(21) Appl. No.: 18/472,472

(22) Filed: Sep. 22, 2023

(51) Int. Cl. C12N 15/86; C12M 1/00; C12M 1/12; C12N 5/071

(52) U.S. Cl. CPC C12N 15/86; C12M 25/14; C12M 29/10; C12N 5/0686; C12N 2509/00; C12N 2510/02; C12N 2513/00; C12N 2740/15043; C12N 2740/15052

(57) ABSTRACT

The design, fabrication and applications of a three-dimensional (3D) bioreactor with filled void structure. The bioreactor comprises non-random voids filled with a non-random internal structure where the voids are interconnected through non-random pore channels. The 3D bioreactor provides a three-dimensional surface area for cell adherence and growth.

### FIELD

The present disclosure relates to the design, fabrication and applications of a three-dimensional (3D) bioreactor with filled void structure. The bioreactor comprises non-random voids filled with a non-random internal structure, where the voids are interconnected through non-random pore channels. The 3D bioreactor provides a continuous three-dimensional surface area for cell adherence and growth. The 3D bioreactor is also scalable with a defined geometry, surface coating and fluidic dynamics that can maintain a monolayer cell culture and reduce or prevent cell aggregation, phenotype change, or extracellular production.

### BACKGROUND

Viral vectors are commonly used by molecular biologists to deliver genetic material into cells. Viral vector manufacturing is currently estimated to grow annually, and the process is reportedly performed inside of an animal (in vivo) or in cell cultures (in vitro).

U.S. Pat. No. 10,988,724 relates to the design, fabrication, and applications of three-dimensional (3D) bioreactors for cell expansion and cell derived substance production. The bioreactor is composed of non-random voids interconnected through non-random pores providing a continuous three-dimensional surface area for cell adherence and growth.

U.S. Pat. No. 11,149,244 also identifies a three-dimensional bioreactor for growth of viral vector producing cells. The reactor is described as having a plurality of voids having a surface area for cell expansion. The voids are said to have a diameter D, a plurality of pore openings between the voids with a diameter d, such that D>d and wherein 90% or more of the voids have a selected void volume (V) that does not vary by more than +/−10% and 90% or more of the pore openings between the voids have a value of d that does not vary by more than +/−10%. The bioreactor is identified as having particular utility to promote T-cell expansion for T-cell based cancer treatment.

U.S. Publication 2021/0317396 relates to the design, fabrication and applications of a three-dimensional bioreactor for expansion of viral vector producing cells and ultimate harvesting of viral vectors. The bioreactor is composed of non-random interconnected voids providing a continuous three-dimensional surface area for cell adherence and growth.

Accordingly, a need remains to provide even further improved 3D bioreactors with increased available surface area and associated methods for cell expansion and growth, and in particular, for expansion of viral vector producing cells.

### SUMMARY

A three-dimensional (3D) bioreactor for growth of cells comprising a biocompatible polymer material have a plurality of voids and a surface for cell expansion having a diameter D in the range of 0.4 mm to 100.0 mm, a plurality of pore channels with openings between the voids having a diameter d in the range of 0.2 mm to 10.0 mm, and a plurality of internal structures positioned within the voids having an internal structure volume (V_{IS}). In addition, 90.0% or more of the voids have a selected volume V that does not vary by more than +/−10.0%, 90.0% or more of the pore openings between the voids have a value of d that does not vary by more than +/−10.0%, and 90.0% or more of the internal structures have an internal structure volume (V_{IS}) that does not vary by more than +/−10.0%.

A method for expansion of cells comprising supplying a three-dimensional (3D) bioreactor comprising a plurality of voids having a surface area for cellular expansion and a plurality of internal structures within said plurality of voids also having a surface area for cell expansion. The plurality of voids have a diameter D including a plurality of pore channels with openings between the voids having a diameter d, such that D>d and wherein 90% or more of the voids have a void volume (V) that does not vary by more than +/−10.0%, 90% or more of the pore openings between the voids have a value of d that does not vary by more than +/−10.0% and 90% of more of the internal structures within the voids have a volume (V_{IS}) that does not vary by more than +/−10.0%. This may then be followed by seeding the three-dimensional (3D) bioreactor with cells and flowing a perfusion media through the three-dimensional (3D) bioreactor and promoting cellular expansion.

### BRIEF DESCRIPTION OF THE DRAWINGS

FIG. 1 provides an initial cross-sectional view of a portion of the 3D bioreactor.

FIG. 2 provides a cross-sectional and segmented view of the 3D bioreactor.

FIG. 3 provides a 2D view of the voids of the 3D bioreactor illustrating the voids and interconnected pore channels.

FIG. 4 provides a 2D cross-sectional view of the internal structures positioned with the voids with the connecting structures.

FIG. 5 provides a segmented cross-sectional view of the 3D bioreactor.

FIG. 6 provides a close-up perspective view of the internal structures and a portion of the connecting structures within the 3D bioreactor.

FIG. 7 shows the 3D bioreactor in a housing with an inlet and outlet compartment for a perfusion system.

FIG. 8 provides a relatively high-resolution image of a portion of the 3D bioreactor herein.

FIG. 9 provides another relatively high-resolution image of a portion of the 3D bioreactor herein.

FIG. 10 provides another relatively high-resolution image of a portion of the 3D bioreactor herein.

FIG. 11A shows the 3D bioreactor herein seeded with 293 FT cells with brightfield imaging.

FIG. 11B shows the 3D bioreactor herein seeded with 294 FT cells with fluorescent imaging.

FIG. 12A shows the wall of a pore in the 3D bioreactor seeded with 293 FT cells.

FIG. 12B shows all of a pore in the 3D bioreactor seeded with 294 FT cells at a different focal level than in FIG. 12A.

FIG. 12C shows seeded 293 FT cells on the 3D bioreactor internal structure surface within the bioreactor voids.

FIG. 12D shows seeded 293 FT cells on the 3D bioreactor internal structure surface within the bioreactor void at a different focal level than in FIG. 12C.

FIG. 13A shows the 3D bioreactor herein after harvesting of 293 FT cells, fluorescent imaging taken during dissociation showing cell clusters dissociated from the 3D bioreactor internal surfaces.

FIG. 13B shows fluorescent imaging of the 3D bioreactor internal void surface after harvesting and washing depicting no cells remaining on the 3D bioreactor internal surfaces.

### DETAILED DESCRIPTION

The present disclosure relates to a bioreactor design and corresponding operating capability to achieve cellular expansion, and in particular, expansion of viral vector producing cells. Reference to a bioreactor herein refers to the disclosed 3D reactor having a plurality of voids, in which biological and/or biochemical processes can be implemented under selected environmental and operating conditions. This initially and preferably includes control of one or more of the following: geometry/size of the voids, interconnected pore channel size between the voids and total number of voids included (determining the overall dimension of the bioreactor) as well as the geometry/size of the internal structures positioned with the voids. In addition, one may selectively control surface coatings, flow characteristics through the voids within the three-dimensional (3D) bioreactor, pH, temperature, pressure, oxygen, nutrient supply, and/or waste removal.

As noted, the 3D bioreactor herein is particularly suitable for the growth of cells and in particular, culturing of HEK 293T cells providing lentiviral vectors. The 3D bioreactor can also extend to produce other types of viruses and vaccines based on live-attenuated viruses, or inactivated viruses, or viral vectors.

More specifically, the three-dimensional (3D) bioreactor includes a continuous interconnected 3D surface area that provides for the ability for the preferred viral vector producing cells to adhere and grow as a monolayer and also defines within the bioreactor a plurality of interconnected non-random voids which are preferably of spherical shape with internal concave surfaces to maximize the surface to volume ratio. A void is understood as an open space of some defined volume. By reference to non-random it should be understood that one can now identify a targeted or selected number of voids in the 3D bioreactor that results in an actual repeating void size and/or geometry of a desired tolerance.

By reference to a continuous surface, it is understood that the expanding viral vector producing cells can readily migrate from one surface area location into another within the 3D bioreactor, and the surface preferably does not include any random interruptions, such as random breaks in the surface or random gaps of 0.1 mm or more. Preferably, 50% or more of the surface area within the 3D bioreactor for cell expansion is a continuous surface, more preferably, 60% or more, 70% or more, 80% or more, 90% or more, 95% or more or 99% or more of the surface area within the 3D bioreactor is continuous.

In addition, the 3D bioreactor includes non-random interconnecting pore channels with openings between the voids. Reference to pore channel may therefore be understood as a conduit for which a fluid such as perfusion media may travel. Again, reference to non-random should be understood that one can now identify a targeted or selected number of pore channels for the voids, of a selected pore channel diameter and length, that results in an actual number of pore channels having pore channel diameters and lengths of a desired tolerance.

The bioreactor herein may preferably be made of biocompatible or bio-inert polymeric materials such as polystyrene, polycarbonate, acrylonitrile-butadiene-styrene (ABS), polylactic acid (PLA), polycaprolactone (PCL) used in FDM (fused deposition modeling) 3D printing technology. Reference to biocompatible or bio-inert should be understood as a material that is non-toxic to the culturing cells. In addition, the polymeric materials for the 3D bioreactor are preferably selected from those polymers that at not susceptible to hydrolysis during cell cultivation, such that the amount of hydrolysis does not exceed 5.0% by weight of the polymeric material present, more preferably it does not exceed 2.5% by weight, and most preferably does not exceed 1.0% by weight. The bioreactor may also be made of biocompatible photosensitive materials (e.g., Pro3Dure, Somos WaterShed XC 11122, etc.) used in SLA (stereolithography) and DLP (digital light processing) 3D printing technologies.

It is preferable that the material used to fabricate the bioreactor is not degradable in aqueous medium and can provide a mechanical stable structure to tolerate aqueous medium flow during cell expansion. It is preferable that the material and manufacturing process results in a solid and relatively smooth interconnected surface area for monolayer cell expansion. By reference to a solid surface, it should be understood that the surface is such that it will reduce or prevent penetration or embedding by the culturing viral vector producing cells, which typically have a diameter of about 20 microns to 100 microns. Preferably, the 3D bioreactor herein is one that has a surface that has a surface roughness value (Ra), which is reference to the arithmetic average of the absolute values of the profile height deviations from the mean line, recorded within an evaluation length. Accordingly, it is contemplated herein that Ra of the 3D bioreactor surface will have a value of less than or equal to 20 μm, more preferably, less than or equal to 5 μm.

The 3D bioreactor herein is also preferably one that is formed from material that indicates a Shore D Hardness of at least 10, or in the range of 10-95, and more preferably in the range of 45-95. In such regard, it is also worth noting that the 3D bioreactor herein is one that preferably does not make use of a hydrogel type structure, which may be understood as a hydrophilic type polymeric structure, that includes some amount of crosslinking, and which absorbs significant amounts of water (e.g., 10-40% by weight). It is also worth noting that the 3D bioreactor herein is one that preferably does not make use of collagen, alginate, fibrin and other polymers that cells can easily digest and undergo remodeling.

Furthermore, the 3D bioreactor herein is preferably one that is made from materials that have a Tensile Modulus of at least 0.01 GPa. More preferably, the Tensile Modulus has a value that is in the range of 0.01 GPa to 20.0 GPa, at 0.01 GPa increments. Even more preferably, the Tensile Modulus for the material for the 3D bioreactor is in the range of 0.01 GPa to 10.0 GPa or 1.0 GPa to 10 GPa. For example, with respect to the earlier referenced polymeric materials suitable for manufacture of the 3D bioreactor herein, polystyrene indicates a Tensile Modulus of about 3.0 GPa, polycarbonate at about 2.6 GPa, ABS at about 2.3 GPa, PLA at about 3.5 GPa and PCL at about 1.2 GPa.

The 3D bioreactor design herein with such preferred regular geometric characteristics and continuous surface area is preferably fabricated by additive manufacturing technologies, such as FDM, selective laser sintering (SLS), stereolithography (SLA), digital light processing (DLP) 3D printing technologies, etc., according to computer generated designs made available by, e.g., a SolidWorks™ computer-aided design (CAD) program.

By way of preferred example, the process utilizing SolidWorks™ to create the 3D bioreactor design herein is described below. A computer model for the bioreactor negative is initially created. More specifically, what may therefore be described as a 3D bioreactor negative was created, e.g., using 6.0 mm diameter spheres with 1.0 mm diameter connecting pore channels between spheres. Of course, other possible dimensions are contemplated within the broad context of this disclosure as described herein.

Moreover, the 3D bioreactor herein now includes filled void structure, namely internal structures positioned and mechanically fixed within the voids that can be of a desired geometrical shape, offering additional surface area for cell expansion, such as preferably in the form or shape of a sphere. However, other geometries are contemplated, including but not limited to cubes, cuboids, cone, or cylinders.

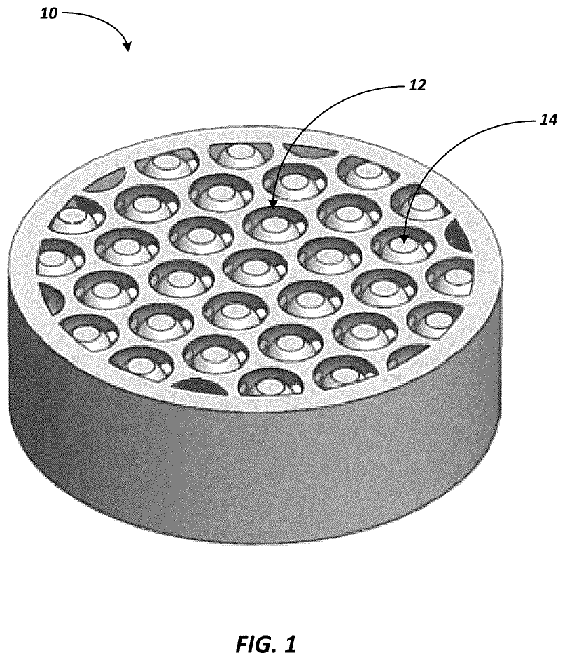

Reference is therefore now made to FIG. 1, which offers an initial cross-sectional view of a portion of the 3D bioreactor 10. As can now be appreciated, the 3D bioreactor 10 includes a plurality of non-random voids 12 that include a plurality of non-random internal structures 14 positioned within the voids. Both the non-random voids and non-random internal structure within the voids provide a surface area for cellular expansion. The internal structures illustrated herein are preferably of a spherical type of shape. The 3D bioreactor herein having non-random voids 12 and non-random internal structures 14 is one that preferably provides cellular expansion from a relatively low number of donor cells, such as viral vector producing HEK 293T cells, that also can reduce or eliminate culture passages and related MSC phenotype alterations.

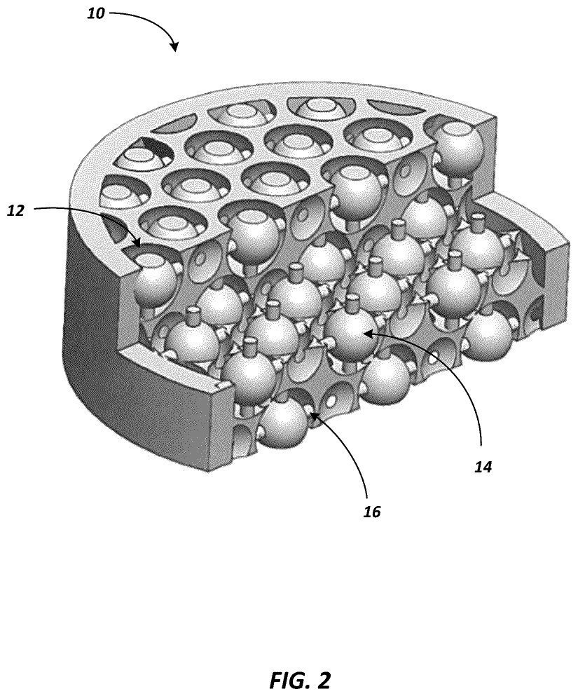

FIG. 2 provides a cross-sectional and segmented view of the 3D bioreactor 10, where the plurality of voids 12 can again be seen along with the plurality of internal structures 14. As can now also be seen, the internal structures 14 are also positioned with the voids 12 and are preferably attached to the void surface so that the internal structures 14 are fixed and suspended within the voids. Accordingly, the internal structures 14 are preferably mechanically fixed within the voids and the outer surface of these fixed internal structures preferably do not impinge and contact with the void surface. This attachment of the internal structures 14 is preferably achieved by a mechanical connecting structure 16 which can also be of a selected non-random geometric shape and is preferably cylindrical. Moreover, for a given internal structure 14, there are preferably at least two connecting structures 16 to connect and engage on one end with the surface of the internal structure 14 and on another end to the void surface. Accordingly, for a given internal structure 14 within a given void 12, there are preferably 2-4 connecting structures to again, as noted, suspend, immobilize and fix the internal structure 14 within a given void.

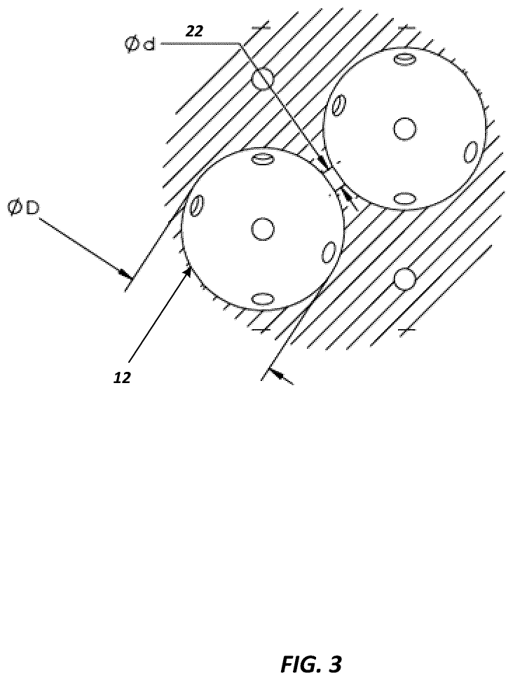

FIG. 3 illustrates in 2D view the voids of the 3D bioreactor and the interconnected and non-random pore channel 22 between the voids 12. As can be seen, the void 22 is identified to have a diameter “D” (ØD). Diameter “D” may therefore be understood as the longest distance between any two points on the internal void surface. The pore channel 22 has a diameter “d” (Ød). Diameter “d” may therefore be understood as the longest distance between any two points on the internal pore channel surface that defines an opening in the pore channel. The pore channel length (L_{pore channel}) may therefore be understood as the distance from one void to the other as spaced apart by the pore channel 22. It may therefore be appreciated that by reducing the value of L_{pore channel} there will be relatively higher packing of the voids within a given 3D bioreactor. The pore channels are also preferably sourced from a variety of geometrical shapes, one of which is preferably a tubular shape.

D preferably has a value between 0.4 mm to 100.0 mm, more preferably, 0.4 mm to 50.0 mm, and also in the range of 0.4 mm to 25.0 mm. One particularly preferred value of D falls in the range of 2.0 mm to 10.0 mm and the preferred value of the pore channel diameter d is in the range of 0.1 mm to 10.0 mm. It is also worth noting that with respect to any selected value of diameter D for the voids in the range of 0.4 mm to 100.0 mm, and any selected value of diameter d for the pores in the range of 0.2 mm to 10.0 mm, the value of D is such that it is greater than the value of d (D>d). The pore channel length (L_{pore channel}) is also preferably in the range of 0.1 mm to 1.0 mm.

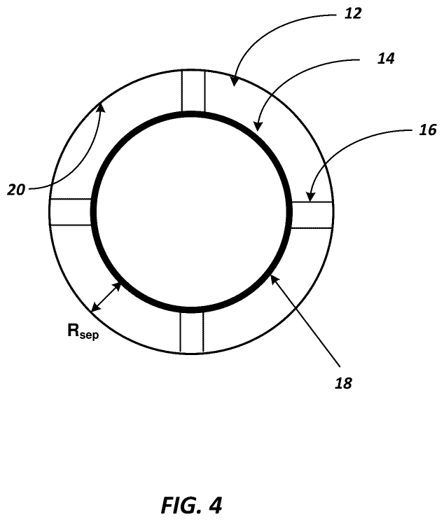

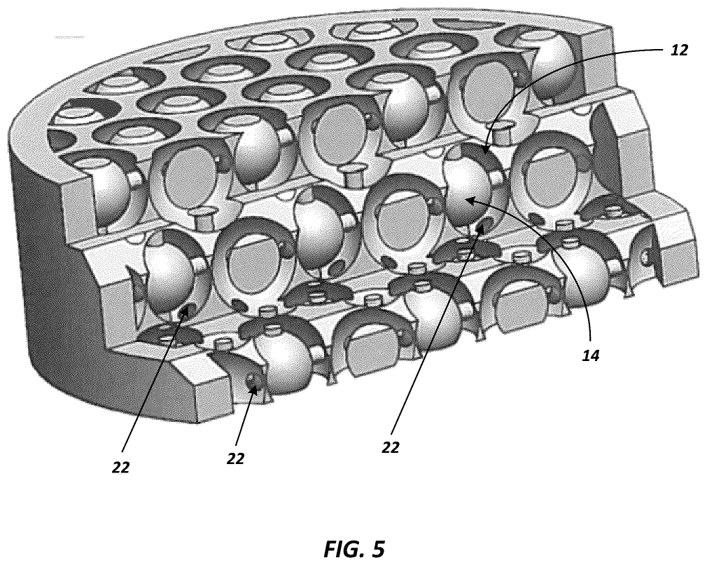

FIG. 4 next provides a 2D cross-sectional view of the internal structures 14 positioned within the voids 12 along with the connecting structures 16. The pore channels 22 are not shown in this cross-section. As can now be appreciated the internal structures 14 are fixed and suspended within the voids by use of connection structure 16 which are connected to the outer surface 18 of the internal structure 14 and to the surface 20 of the voids 12. As noted above, since the 3D bioreactor herein is preferably produced by additive manufacturing, it should be appreciated that the connecting structures 16 are preferably of a non-random unitary type structure within the entirety of the structure of the 3D bioreactor. It should also now be appreciated that the internal structures 14 within the voids serve to increase the available surface area within the 3D bioreactor for cell adherence and growth. FIG. 5 is another segmented cross-sectional view of the 3D bioreactor herein where one can again see the voids 12 along with the internal structures 14. In addition, the plurality of non-random pore channels can be seen at 22.

Preferably, at least 90.0% to 100% of the voids present in the 3D bioreactor have at least 2 pore channel openings per void. More preferably, at least 90.0% to 100% of the voids in the 3D bioreactor have 8-12 pore channel openings per void. In one particularly preferred embodiment, at least 90.0% to 100% of the voids in the 3D bioreactor have 12 pore channel openings per voids between adjacent voids within the plurality of voids present, and more preferably, there are 8-12 interconnected pore channel openings between the adjacent voids, and in one particularly preferred embodiment, there are 12 pore channel openings between the adjacent voids.

It can now be appreciated that the 3D bioreactor herein can be characterized with respect to its non-random characteristics. Preferably, all of the voids within the 3D bioreactor are such that they have substantially the same volume to achieve the most efficient 3D space packing and offer the largest corresponding continuous surface area. With respect to the total number of interconnected voids present in any given 3D bioreactor, preferably, 90.0% or more of such voids, or even 95.0% or more of such voids, or even 99.0% to 100% of such voids have a void volume (V) whose tolerance is such that it does not vary by more than +/−10.0%, or +/−5.0%, or +/−2.5% or +/−1.0%, or +/−0.5% or +/−0.1%.

Another non-random characteristic of the 3D bioreactor herein are the pore channel openings between the voids, having a diameter d (see again FIGS. 3 and 5). Similar to the above, 90.0% or more of the pore channel openings, or even 95.0% or more of the pore channel openings, or even 99.0% to 100% of the pore channel openings between the voids, indicate a value of d whose tolerance does not vary more than +/−10. %, or +/−5.0%, or +/−2.5% or +/−1.0%, or +/−0.5% or +/−0.1%. In addition, with respect to the length of the pore channels (L_{pore channel}) with the 3D bioreactor, 90.0% of the pore channel lengths, or even 95.0% or more of the pore channel lengths, or even 99.0 to 100% of the pore channel lengths between the voids, have a value whose tolerance does not vary by more than +/−10. %, or +/−5.0%, or +/−2.5% or +/−1.0%, or +/−0.5% or +/−0.1%.

It can therefore now by appreciated that the 3D bioreactor herein for growth of cells comprises a surface area for cell expansion, a plurality of voids having a diameter D (the longest distance between any two points on the internal void surface) and a plurality of pore channel openings between the voids having a diameter d (the longest distance between any two points on the internal pore channel surface that defines an opening in the pore channel), where D>d. In addition, 90% or more of the voids have a void volume (V) that does not vary by more than +/−10.0%, and 90% or more of the pore channel openings have a value of d that does not vary by more than +/−10.0%. The voids as noted may preferably be of spherical shape and preferably define an internal concave surface for cellular expansion.

The additional non-random characteristic of the 3D bioreactor herein applies to the internal structures 14, the connecting structures 16 and the pore channels 22. Namely, the internal structures 14, and/or connecting structures 16 and/or the pore channels 22, are such that they preferably define substantially the same volume to achieve the most efficient 3D space packing and offer the largest corresponding continuous surface area for cellular expansion. With respect to the total number of internal structures 14 and/or connecting structures 16 and/or pore channels 22 in any given 3D bioreactor, preferably, 90.0% or more of such internal structures 14 and/or connecting structures 16 and/or pore channels 22, or even 95.0% or more of such internal structures 14 and/or connecting structures 16 and/or pore channels 22, or even 99.0% to 100% of such internal structures 14 and/or connecting structures 16 and/or pore channels 22, define an internal structure volume (V_{IS}), a connecting structure volume (Vₑₛ) and a pore channel volume (V_{PC}). The tolerance is such that V_{IS} and/or Vₑₛ and/or V_{PC} preferably do not vary by more than +/−10.0%, or +/−5.0%, or +/−2.5% or +/−1.0%, or +/−0.5% or +/−0.1%.

Another preferred characteristic of non-random internal structures 14 is that they indicate a radial separation (Rₛₑₚ) between the outer surface 18 of the internal structure 14 and the surface 20 on the voids 12 (see again, FIG. 4). This radial separation (Rₛₑₚ) preferably has a value in the range of 0.25 mm to 1.00 mm. More preferably, the radial separation (Rₛₑₚ) has a value of 0.25 mm, 0.30 mm, 0.35 mm, 0.40 mm, 0.45 mm, 0.50 mm, 0.55 mm, 0.60 mm, 0.65 mm, 0.70 mm, 0.85 mm, 0.90 mm, 0.95 mm, and 1.00 mm. Moreover, the radial separation value is another non-random characteristic, and for a given radial spacing, such as a radial spacing in the preferred range of 0.25 mm to 1.00 mm, the value is such that it does not vary by more than +/−10.0%, or +/−5.0%, or +/−2.5% or +/−1.0%, or +/−0.5% or +/−0.1%. Therefore, by way of example, the radial separation (Rₛₑₚ) within the 3D bioreactor herein may preferably have a value of 0.50 mm, where such value does not vary by more than +/−10.0%, or +/−5.0%, or +/−2.5% or +/−1.0%, or +/−0.5% or +/−0.1%.

Accordingly, it may be appreciated that given the radial separation (Rₛₑₚ) as preferably falling in the range of 0.25 mm to 1.0 mm, and the feature that the void diameter D preferably has a value between 0.4 mm to 100.0 mm, the diameter of the internal structure (D_{IS}) 14 is then preferably selected to maintain such radial separation within the bioreactor. For example, if the radial separation is to be 0.25 mm, and the void volume is to be 0.4 mm, then the internal structure can have a diameter (D_{IS}) of 0.15 mm. Similarly, if the radial separation is to be 0.25 mm, and the void volume is to be 100.0 mm, then the internal structure can have a diameter (D_{IS}) of 99.75 mm. Or, if the radial separation is to be 0.50 mm, and the void volume is to be 0.4 mm, then the internal structure can have a diameter (D_{IS}) of 0.35 mm. Or, if the radial separation is to be 0.50 mm, and the void volume is to be 100.0 mm, then the internal structure can have a diameter (D_{IS}) of 99.50 mm.

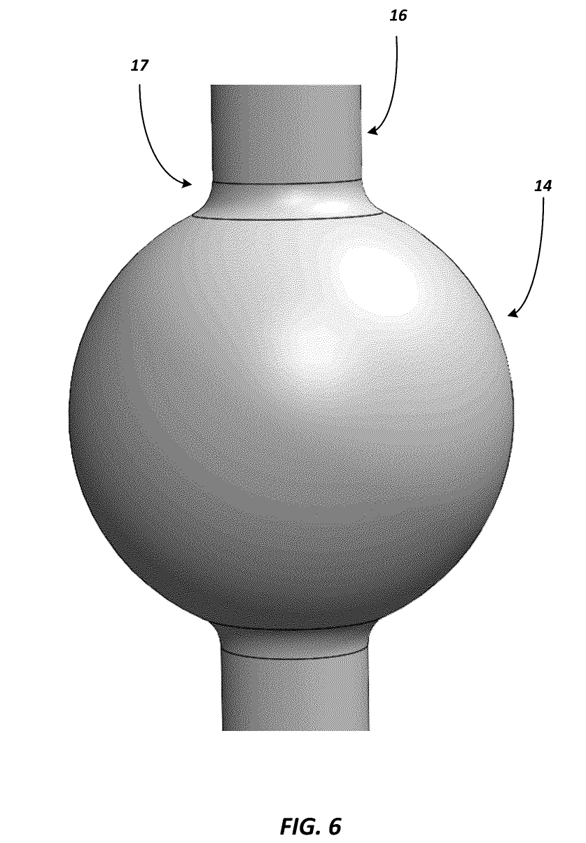

FIG. 6 provides a close-up perspective view of the internal structure 14 and a portion of the connecting structures 16. As can be observed, preferably, there is a transition structure 17 between the internal structure 14 and the connecting structure 16. Where the internal structure 14 (IS) has a diameter D_{IS}, and the connecting structure (CS) 16 has a diameter D_{CS}, the following is the preferred relationship of their values:

$${D}_{IS} * 0.25 \leq {D}_{CS} \geq {D}_{IS} * 0.5$$

The 3D bioreactor herein including the voids 12 with internal structure 14 is such that the surface area-to-volume ratio is driven by, and inversely correlated to, the diameter D of the voids and the diameter of the internal structure (D_{IS}). The values of D and Dis may be controlled herein by the preferred manufacture method of additive manufacturing along with a consideration of the surface area made available with the 3B bioreactor relative to the size of the cells that may be attached to the 3D bioreactor internal surface.

One particular and preferred goal of this application is to create a relatively large surface area for growing cells. To mimic a 2D system, it is preferred that the internal surface of the 3D bioreactor is relatively flat, particular for cells having a size of 20 μm to 100 μm. Accordingly, it is preferred that the curvature of the internal surfaces of the 3D bioreactor herein should be reduced, meaning that the diameter of the voids is set relatively high for the cells at issue.

Another consideration for the design of the 3D bioreactor herein is to preferably reduce or prevent cell aggregation inside the bioreactor as cell aggregation can cause cell differentiation instead of cell proliferation. Preferably, to reduce or avoid cell aggregation, the diameter (d) of the pores channels (22) is a minimum of 500 μm (0.50 mm) or in the range of 0.5 mm to 10.0 mm and the diameter D of the voids are greater than 1.0 mm or in the range of greater than 1.0 mm to 100.0 mm, or even more preferably the diameter D of the voids is in the range of greater than 1.0 mm to 10.0 mm. As noted above, under these more preferred parameters for the diameter D of the voids, the internal structures preferably have a diameter that maintains a radial separation (Rₛₑₚ) that again falls in the range of 0.25 mm to 1.0 mm.

For the 3D printed bioreactor herein using, e.g., ABS, the hydrophobic internal surfaces of the bioreactor is preferably modified to allow for cell adherence. Preferably one may utilize polydopamine as a primer coating which may be followed with a fibronectin coating to provide hydrophilic internal surfaces. It should also be noted, that the polydopamine primer coating can be combined with other coatings such as peptides, collagen, laminin, multiple cell extracellular matrix proteins, or selected antibodies that are required by a particular cell type. After polydopamine is deposited on the bioreactor surface, it can then bind with functional ligands via Michael addition and/or Schiff base reactions. The ligand molecules therefore preferably include nucleophilic functional groups, such as amine and thiol functional groups. One may also utilize plasma treatment to alter the internal surface properties to modify a hydrophobic internal 3D bioreactor surface herein and provide a relatively hydrophilic internal surface. The plasma treatment may preferably take place in the presence of oxygen.

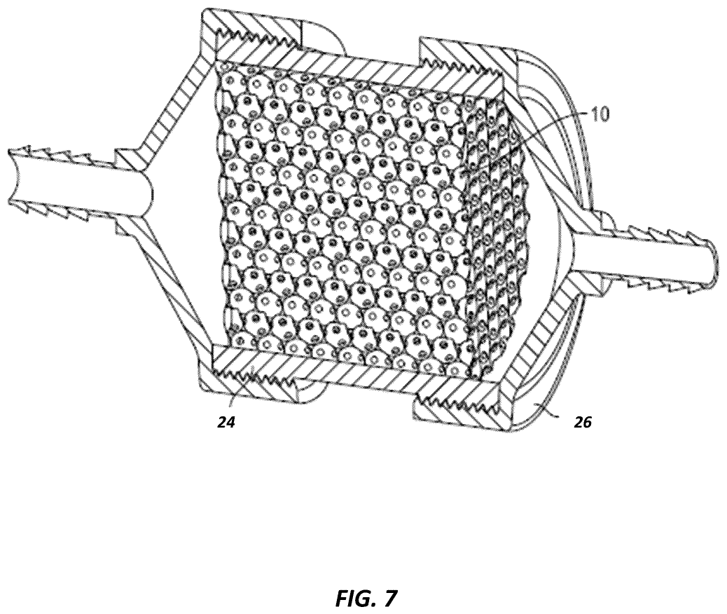

The bioreactor 10 herein with voids 12 and internal structure 14 and pore channels 22 may therefore preferably serve as the 3D bioreactor as further illustrated in FIG. 7. More specifically, the bioreactor 10 may preferably be positioned in a housing 24 and then placed in the inlet and outlet compartment 26 for which inflow and outflow of fluid may be provided. Preferably, the bioreactor 10, housing 24, and the inlet and outlet compartment 26 can be fabricated as a single component using Additive Manufacturing technology. As shown in FIG. 7, the 3D bioreactor 10 in housing 24 and inlet and outlet compartment 26 may become part of an overall 3D bioreactor system for cellular expansion.

More specifically, the 3D bioreactor is preferably positioned within a perfusion system which delivers a viral vector producing cell culture medium and oxygen through the 3D bioreactor for promoting such cell growth. Multiple passage cell expansion methods used in 2D T-flask can also be directly applied to the 3D bioreactor except a 3D bioreactor has the cell culture area equivalent to 10s, 100s, or 1000s of T-flasks. Besides multiple passage cell expansion, a one-step expansion from a low number of donor cells to a clinically relevant number of cells is contemplated thus eliminating the multiple-passaging problem that induces MSC phenotype changes during expansion.

### WORKING EXAMPLES

### Example 1

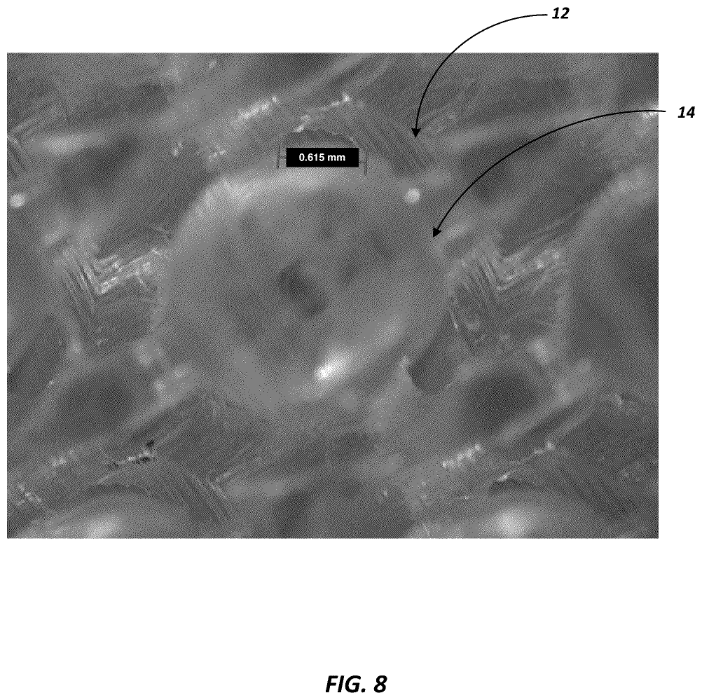

A 3D bioreactor herein having internal voids 12 and internal structure 14 was prepared by additive manufacturing (SLA 3D printing) and images were taken with a relatively high resolution Liecia camera (21X). See FIG. 8. The pore channel diameter d appeared to have a value of 0.615 mm.

### Example 2

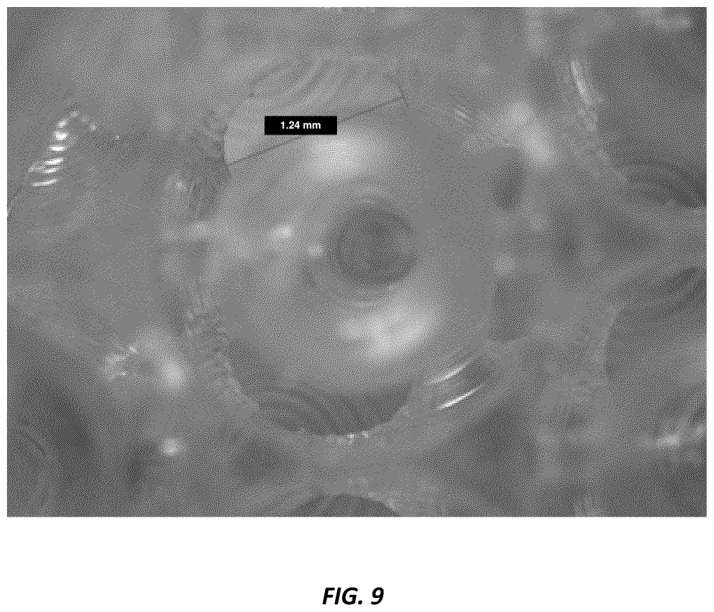

A 3D bioreactor herein again having internal voids and internal structure was prepared by additive manufacturing (SLA 3D printing) and the pore channel diameter appeared to have a value of 1.24 mm. See FIG. 9.

### Example 3

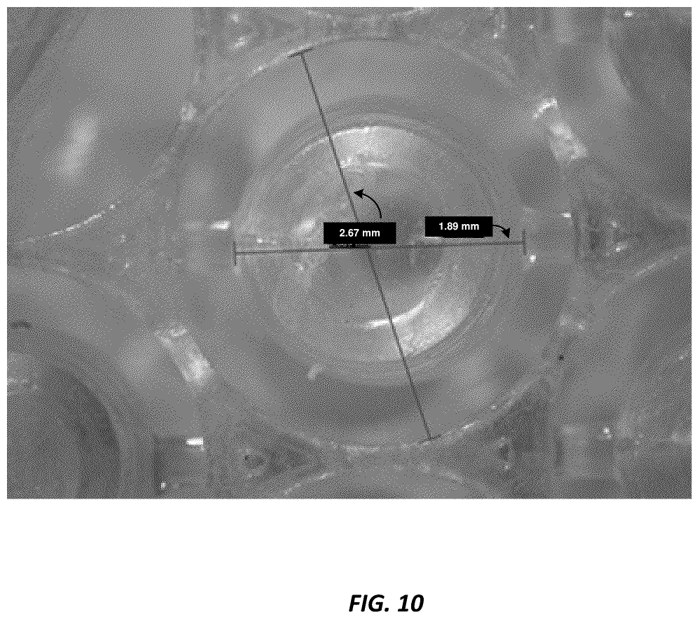

A 3D bioreactor herein again having internal voids and internal structure was prepared by additive manufacturing (SLA 3D printing) See FIG. 10. The internal voids had a diameter D of 2.67 mm and the diameter of the internal structures (D_{IS}) was 1.89 mm.

### Example 4

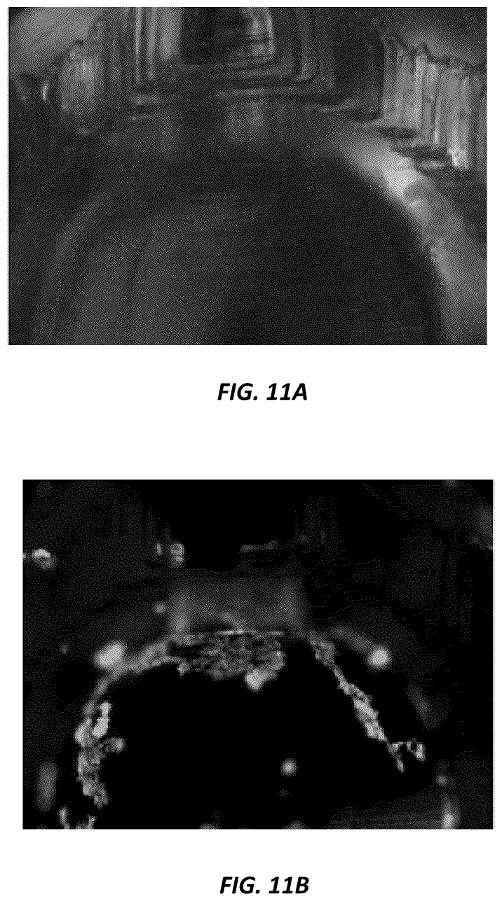

Reference is made to FIG. 11A which shows the 3D bioreactor herein seeded with 293 FT cells, with brightfield imaging to show the internal structure surface. FIG. 11B applies fluorescent imaging at the same location depicting the cells growing on the surface of the internal structure surface within the 3D bioreactor voids.

### Example 5

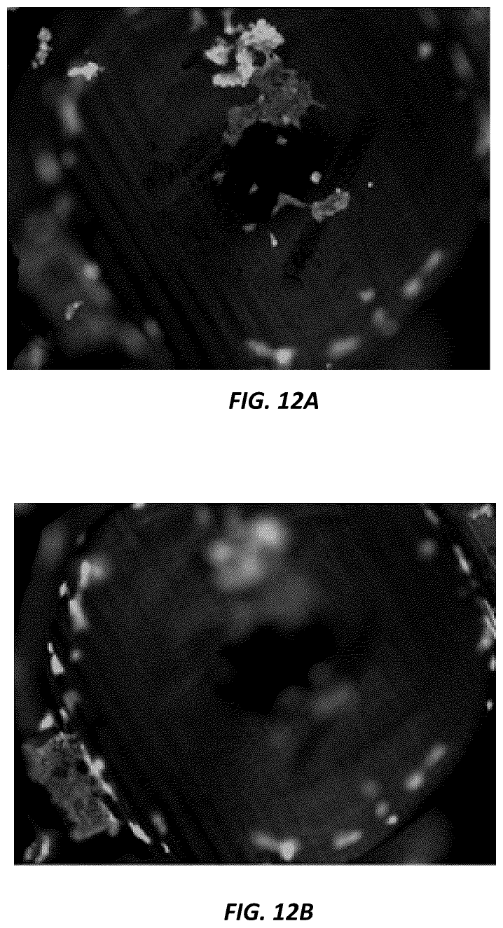

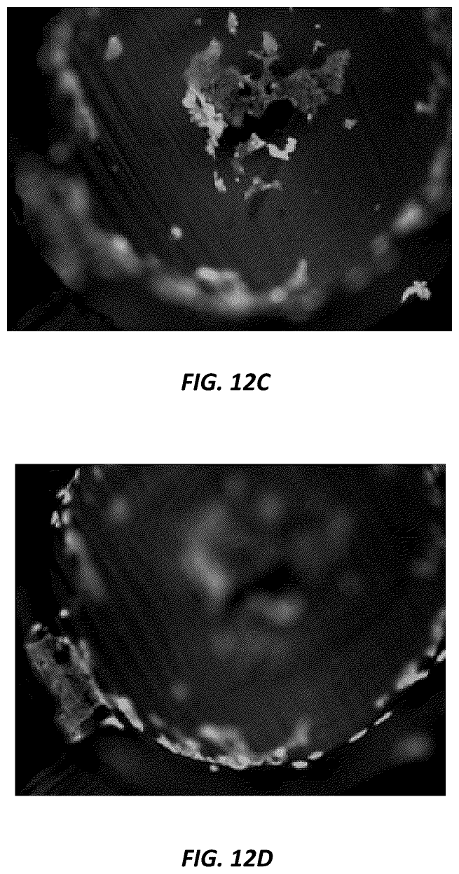

Reference is made to FIGS. 12A, 12B, 12C and 12D. Specifically, FIGS. 12A and 12B shows the wall of the same pore of the 3D bioreactor, seeded with 293 FT cells, at different focal levels. FIGS. 12C and 12D show the seeded cells on the internal structure surface within the 3D bioreactor voids, again, at different focal levels.

### Example 6

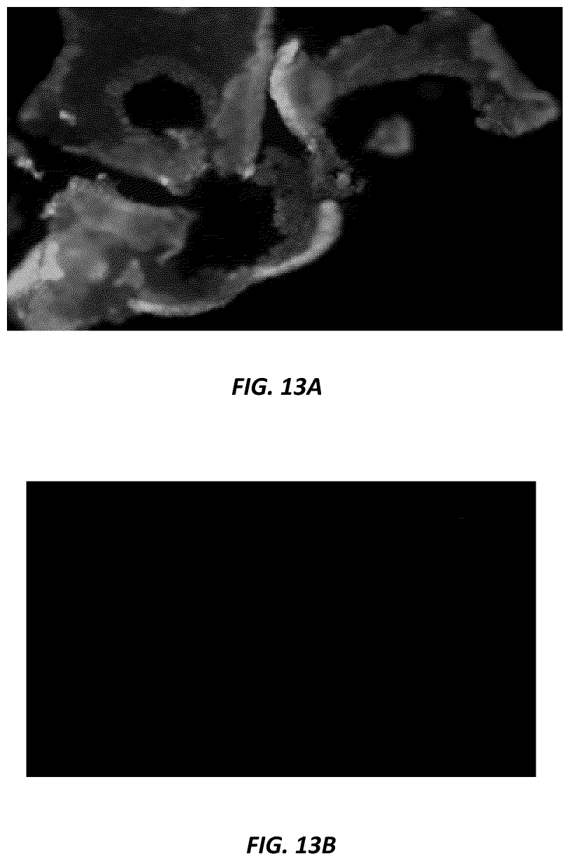

This example was conducted to demonstrate the ability to detach cells from the 3D bioreactor herein after expansion. Accordingly, the 293 FT cells were tested for cell detachment with Trypsin-EDTA (0.25%). Trypsin was used with a 5 minute (37° C.) incubation period and was successful in detachment of the subject cells. FIG. 13A shows the 3D bioreactor herein after harvesting of 293 FT cells, fluorescent imaging taken during dissociation showing cell clusters dissociated from the 3D bioreactor internal surfaces. FIG. 13B shows fluorescent imaging of the 3D bioreactor internal void surface after harvesting and washing depicting no cells remaining on the 3D bioreactor internal surfaces.

What is claimed is:

1. A three-dimensional (3D) bioreactor for growth of cells comprising:
a biocompatible polymer material having a plurality of voids and a surface for cell expansion having a diameter D in the range of 0.4 mm to 100.0 mm;
a plurality of pore channels with openings between said voids having a diameter d in the range of 0.2 mm to 10.0 mm;
a plurality of internal structures positioned within said voids having an internal structure volume (V_{IS});
wherein (a) 90.0% or more of said voids have a selected volume V that does not vary by more than +/−10.0%; (b) 90.0% or more of said pore channel openings between said voids have a value of d that does not vary by more than +/−10.0%; and (c) 90.0% or more of said internal structures have an internal structure volume (V_{IS}) that does not vary by more than +/−10.0%.

2. The three-dimensional (3D) bioreactor of claim 1 wherein said internal structures have an outer surface, said voids have a surface, wherein there is a radial separation (Rₛₑₚ) between said outer surface of said internal structures and said surface of said voids.

3. The three-dimensional (3D) bioreactor of claim 2 wherein said radial separation (Rₛₑₚ) has a value that does not vary by more than +/−10.0%.

4. The three-dimensional (3D) bioreactor of claim 2 wherein said radial separation (Rₛₑₚ) has a value in the range of 0.25 mm to 1.00 mm.

5. The three-dimensional (3D) bioreactor of claim 1 further including a plurality of connecting structures which connect to said void surface and to said internal structures positioned within said voids.

6. The three-dimensional (3D) bioreactor of claim 5 wherein said connecting structures define a connecting structure volume (V_{cs}) and said connecting structure volume (V_{cs}) does not vary by more than +/−10.0%.

7. The three-dimensional (3D) bioreactor of claim 5 wherein there are 2-4 connecting structures within a void.

8. The three-dimensional (3D) bioreactor of claim 5 wherein said internal structure has a diameter (D_{IS}) and said connecting structures have a diameter (D_{CS}) and the following relationship applies:
$${D}_{IS} * 0.25 \leq {D}_{CS} \geq {D}_{IS} * 0.5 .$$

9. The three-dimensional (3D) bioreactor of claim 1 wherein said voids have a shape selected from the group consisting of spheres, cubes, cuboids, or cylinders.

10. The three-dimensional (3D) bioreactor of claim 1 wherein said voids have an internal concave surface.

11. The three-dimensional (3D) bioreactor of claim 1 wherein said pore channels have a length (L_{pore channel}) between said voids and L_{pore channel} is 0.1 mm to 1.0 mm.

12. The three-dimensional (3D) bioreactor of claim 1 wherein said pore channels have a length (L_{pore channel}) between said voids and 90% or more of the pore channel lengths have a value that does not vary by more than +/−10.0%.

13. A method for expansion of cells comprising:
supplying a three-dimensional (3D) bioreactor comprising a plurality of voids having a surface area for cellular expansion and a plurality of internal structures within said plurality of voids also having a surface area for cell expansion;
said plurality of voids having a diameter D including a plurality of pore channels with openings between said voids having a diameter d, such that D>d and wherein:
(a) 90% or more of said voids have a void volume (V) that does not vary by more than +/−10.0%;
(b) 90% or more of said pore openings between said voids have a value of d that does not vary by more than +/−10.0%;
(c) 90% of more of said internal structures within said voids have a volume (V_{IS}) that does not vary by more than +/−10.0%;
seeding said three-dimensional (3D) bioreactor with cells and flowing a perfusion media through said three-dimensional (3D) bioreactor and promoting cellular expansion.

14. The method of claim 13 wherein said internal structures have an outer surface, said voids have a surface, wherein there is a radial separation (Rₛₑₚ) between said outer surface of said internal structures and said surface of said voids.

15. The method of claim 14 wherein said radial separation (Rₛₑₚ) has a value that does not vary by more than +/−10.0%.

16. The method of claim 14 wherein said radial separation (Rₛₑₚ) has a value in the range of 0.25 mm to 1.00 mm.

17. The method of claim 13 further including a plurality of connecting structures which connect to said void surface and to said internal structures positioned within said voids.

18. The method of claim 17 wherein there are 2-4 connecting structures within a void.

19. The method of claim 17 wherein said internal structure has a diameter (D_{IS}) and said connecting structure has a diameter (D_{CS}) and the following relationship applies:
$${D}_{IS} * 0.25 \leq {D}_{CS} \geq {D}_{IS} * 0.5 .$$

20. The method of claim 13 wherein said voids have a shape selected from the groups consisting of spheres, cubes, cuboids, or cylinders.

21. The method of claim 13 wherein said voids have an internal concave surface.

22. The method of claim 13 comprising seeding said three-dimensional (3D) bioreactor with viral vector producing cells and flowing a perfusion medium through said three-dimensional (3D) bioreactor and promoting viral vector cell expansion.

23. The method of claim 22 further comprising delivery of a transfection reagent to said viral vector producing cells in said three-dimensional (3D) bioreactor and producing a viral vector.

24. The method of claim 22 wherein said viral vector cells comprise HEK 293T cells and said viral vector comprises a lentiviral vector.
